(19) United States
(12) Patent Application Publication
(10) Pub. No.: US 2014/0275631 A1
(43) Pub. Date: Sep. 18, 2014

(54) Process for synthesizing GGA and its derivatives

(71) Applicant: COYOTE PHARMACEUTICALS, INC., MENLO PARK, CA (US)

(72) Inventors: Obsidiana Abril-Horpel, San Ramon, CA (US); William Haag, San Ramon, CA (US); Ann Pierson, San Ramon, CA (US)

(21) Appl. No.: 13/815,805

(22) Filed: Mar. 15, 2013

(51) Int. Cl. C07C 45/42; C07C 45/29

(52) U.S. Cl. CPC C07C 45/42; C07C 45/29
USPC 568/409; 568/485

(57) ABSTRACT

This invention relates to processes for synthesizing GGA or GGA derivatives and intermediates involved therein.

### FIELD OF THE INVENTION

This invention relates generally to geranylgeranyl acetone (GGA) or GGA derivatives and processes for their syntheses.

### STATE OF THE ART

Geranylgeranylacetone (GGA) is an acyclic isoprenoid compound with a retinoid skeleton having the formula:

[chemical structure]

GGA is a known anti-ulcer drug used commercially and is reported to have neuroprotective and related effects. See, for example, PCT Pat. App. Pub. No. WO 2012/031028 and PCT Pat. App. No. PCT/US2012/027147, each of which is incorporated herein by reference in its entirety. Alternate methods for synthesizing GGA are provided herein.

### SUMMARY OF THE INVENTION

In various aspects, provided herein are processes of syntheses of GGA and derivatives thereof, and intermediates thereto.

In one aspect, a process for preparing a compound of formula (I) is provided,

[chemical structure]

said process comprising:
hydrolyzing a compound of formula (II):

[chemical structure]

wherein:
X and Y are each independently OR⁶, SR⁶, or X and Y together with the carbon atom they are attached to form a ring of formula:

[chemical structure]

wherein each R⁶ is independently C₁-C₆ alkyl,
each X¹ and X² are independently O, or S; q is 1 or 2; each X³ is independently C₁-C₆ alkyl; t is 0, 1, 2, or 3, and
each of R¹, R², R³, R⁴, and R⁵ is independently H or C₁-C₆ alkyl or R¹ and R² together with the carbon atom they are joined to form a C₅-C₆ cycloalkyl optionally substituted with 1-3 C₁-C₆ alkyl.

In further aspects, a process for preparing a compound of formula (II) is provided:

[chemical structure]

said process comprising:
contacting a compound of formula (III):

[chemical structure]

wherein:
the variables are defined as in formula (II) above
with a compound of formula:

[chemical structure]

wherein L is P(R^{z})₃, P(O)(R^{z})₂, SO₂R^{z}, Si(R^{z})₃, preferably P(R^{z})₃; and wherein is R^{z} is a C₁-C₆ alkyl group or an aryl group;
under conditions suitable for olefination of compound of formula (III) to produce a compound of formula (II).

In further aspects, a process for preparing a compound of formula (XXI) is provided:

[chemical structure]

said process comprising:
oxidizing a compound of formula (XXII):

[chemical structure]

In still further aspects, a process for preparing a compound of formula (XXII)

[chemical structure]

said process comprising:
reducing a compound of formula (XXIII)

[chemical structure]

In further aspects, a process for preparing a compound of formula (XXIII):

[chemical structure]

said process comprising:
contacting an orthoacetate of formula R⁷CH₂—C(OR¹⁰)₃ wherein R¹⁰ is C₁-C₆ alkyl with a compound of formula (XXIV):

[chemical structure]

In further aspects, a process for preparing a compound of formula (XXIV):

[chemical structure]

said process comprising:
contacting of a compound of formula (XXV):

[chemical structure]

with a compound of formula

[chemical structure]

In each of the embodiments above, X and Y are as defined in formula (II) above, R⁷ is independently hydrogen or C₁-C₆ alkyl, n is 1-5, R is hydrogen or C₁-C₆ alkyl, preferably an alkyl group, and R¹⁰ is C₁-C₆ alkyl.

### DETAILED DESCRIPTION

It is to be understood that this invention is not limited to particular embodiments described. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to “an excipient” includes a plurality of excipients.

### DEFINITIONS

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 10%, 5%, or 1%.

The term “halo” or “halo group” refers to fluoro, chloro, bromo and iodo.

“Geometrical isomer” or “geometrical isomers” refer to compounds that differ in the geometry of one or more olefinic centers. “E” or “(E)” refers to the trans orientation and “Z” or “(Z)” refers to the cis orientation.

Geranylgeranyl acetone (GGA) refers to a compound of the formula:

[chemical structure]

wherein compositions comprising the compound are mixtures of geometrical isomers of the compound.

The 5-trans isomer of geranylgeranyl acetone refers to a compound of the formula:

[chemical structure]

wherein the number 5 carbon atom is in the 5-trans (5E) configuration.

The 5-cis isomer of geranylgeranyl acetone refers to a compound of the formula:

[chemical structure]

wherein the number 5 carbon atom is in the 5-cis (5Z) configuration.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

As used herein, Cₘ-Cₙ, such as C₁-C₁₀, C₁-C₆, or C₁-C₄ when used before a group refers to that group containing m to n carbon atoms.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). In some embodiments, the term “alkyl” refers to substituted or unsubstituted, straight chain or branched alkyl groups with C₁-C₁₂, C₁-C₆ and preferably C₁-C₄ carbon atoms.

The term “cycloalkyl” refers to a monovalent, preferably saturated, hydrocarbyl mono-, bi-, or tricyclic ring having 5-6 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopentyl, cyclohexyl, and the like. The condensed rings may or may not be non-aromatic hydrocarbyl rings provided that the point of attachment is at a cycloalkyl carbon atom. For example, and without limitation, the following is a cycloalkyl group:

[chemical structure]

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

[chemical structure]

The term “hydrolyzing” refers to adding water across a C—O and/or a C—S bond, such as hydrolyzing a ketal, a thioketal and the likes to the corresponding ketone. A hydrolyzing is performed using various methods well known to the skilled artisan, non limiting examples of which include acidic hydrolysis. A variety of acids such as protic acids and Lewis acids can be used for the hydrolysis.

The term “oxidizing” or “oxidation” refers to taking one or more electron away from a bond or an atom, preferably taking two electrons away from a bond or an atom. Non-limiting examples of oxidation include conversion of an alcohol to an aldehyde.

The term “reducing” or “reduction” refers to adding one or more electron across a bond or an atom, preferably adding two electrons to a bond or an atom. Non-limiting examples of reduction include conversion of a carboxylic acid or an ester thereof to an alcohol.

The term “olefination” refers to conversion of a bond to the corresponding olefinic derivative. For example, without limitation, conversion of C═O to C═CR^{x}R^{y}, wherein R^{x} and R^{y} are alkyl groups.

The term “salt” refers to an ionic compound formed between an acid and a base. When the compound provided herein contains an acidic functionality, such salts include, without limitation, alkai metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds provided and/or utilized herein contain basic functionality, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

### GGA and GGA Derivatives

In various aspects, provided herein are processes for preparing compound of formula (I) and derivatives thereof, and intermediates used in their synthesis, such as those of formulas (II)-(XXIV).

[chemical structure]

[chemical structure]

or a salt thereof, wherein the variables in the structures (I)-(XVIV) are defined as in formula (II) above.

In one embodiment, X and Y together with the carbon atom they are attached to form a ring of formula:

[chemical structure]

wherein each X¹ and X² are independently O, or S; q is 1 or 2; each X³ is independently C₁-C₆ alkyl; and t is 0, 1, 2, or 3.

In one aspect, the GGA derivative has the formula XIX:

[chemical structure]

or a tautomer or pharmaceutically acceptable salt thereof, wherein X, Y, R¹ and R² are as defined herein; and
n is 1, 2, 3, 4 or 5.

In another aspect, the GGA derivative has the formula XX:

[chemical structure]

or a tautomer or pharmaceutically acceptable salt thereof, wherein R¹, R², and n are as defined herein.

In another aspect, the GGA derivative has the formulas (XXI)-(XIV):

[chemical structure]

or a tautomer or pharmaceutically acceptable salt thereof, wherein:
X and Y are each independently OR⁶, SR⁶, or X and Y together with the carbon atom they are attached to form a 5-7 membered heretocyclic ring having 2 oxygen and/or sulfur atoms and optionally substituted with 1-3 C₁-C₆ alkyl groups,
each of R⁶, R⁷ and R⁸ is independently H or C₁-C₆ alkyl; and
n is an integer from 1 to 5.

In one embodiment, X and Y together with the carbon atom they are attached to form a cyclic ketal with two oxygen, two sulfur or one oxygen and one sulfur atom.

In one embodiment, X and Y together with the carbon atom they are attached to form a dioxolane, oxathiolane, dithiolane, dioxane, oxathiane or a dithiane ring.

This invention provides processes for the syntheses of GGA derivatives, such as those of formulas (I)-(XXIV), cis-trans isomers, and sub-formulas thereof.

In one aspect, a process for preparing the GGA derivative of formula (I) is provided. The process comprises contacting a compound of formula (II) with an acid catalyst under conditions suitable for hydrolysis of compound of formula (II) to produce a compound of formula (I).

[chemical structure]

In one embodiment, the conditions suitable for hydrolysis of compound of formula (II) to produce a compound of formula (I) include contacting the compound of formula (II) with an acid catalyst in an inert solvent at a suitable temperature. In one embodiment, the acid catalyst utilized in the process is selected from an aqueous acetic acid, formic acid, trifluoroacetic acid, sulfuric acid, hydrochloric acid, methanesulfonic acid, alkyl or aralkylsulfonic acid or a Lewis acid. The acid is preferably used in catalytic amount.

In another aspect, a process for preparing the GGA derivative of formula (II) is provided. The process comprises subjecting a compound of formula (III) to an olefination reaction under conditions suitable for of compound of formula (III) to produce a compound of formula (II).

[chemical structure]

In one embodiment, the olefination is conducted using Wittig reaction. Wittig reaction or Wittig olefination refers to the reaction of a carbonyl compound, e.g. an aldehyde or a ketone, with a phosphonium ylide to an alkene. Typical Wittig reaction includes deprotonating a phosphonium salt by a base to form a phosphorane and reacting it with the aldehyde. The phosphonium salts or Wittig reagents utilized in the reaction can be obtained by reacting a phosphine, e.g., triphenylphosphine, with a primary or secondary halide under heated conditions, in the presence or absence of a solvent.

In one embodiment, the conditions suitable for olefination of compound of formula (II) to produce a compound of formula (II) include, for example, reacting the aldehyde of formula (III) with a phosphorane in a suitable solvent in the presence of a base. In one embodiment, the olefination of compound of formula (III) includes contacting compound (III) with a Wittig reagent, e.g., C(R¹R²)═P(R^{z})₃ wherein R^{z} is e.g., triphenyl group. In some embodiments, a composition comprising the compound of formula (III) with a Witting reagent e.g., C(R¹R²)═PR^{z}, is provided. Suitable solvents include aliphatic or aromatic hydrocarbons, such as e.g., hexane, benzene or toluene, and ethers such as for example diethyl ether and tetrahydrofuran, or amides, such as e.g., dimethylformamide or hexamethylphosphoric acid triamide. In some cases alcohols or dimethyl sulphoxide can be used as solvent. Suitable bases for the Wittig reaction include metal alcoholates, such as for example sodium ethanolate, metal hydrides, such as for example sodium hydride, metal amides, such as e.g., sodium amide and organometallic compounds, such as for example phenyllithium or butyllithium. The vinyl group in the compound of formula (II) can be formed with specificity and positional selectivity.

In one embodiment, the olefination is conducted using Wittig-Horner reaction. In other embodiments, the olefination is conducted using Peterson olefination reaction. Conditions suitable for these reactions will be apparent to one skilled in the art. For example, the Witting-Horner reaction can be conducted utilizing the Wittig reagent, as described for the Wittig reaction, but with lithium bases, such as e.g., n-butyl lithium, and at low temperatures. In Peterson olefination, for example, α-silylated carbanion is added to the carbonyl compound of formula (III) to give rise to two diastereomeric β-hydroxysilanes, which can be isolated and separately transformed further to alkenes.

In yet another aspect, a process for preparing the GGA derivative of formula (III) is provided. The process comprises oxidizing a compound of formula (IV) under suitable conditions to produce a compound of formula (III).

[chemical structure]

In another aspect, a process for preparing the GGA derivative of formula (VII) is provided. The process comprises oxidizing a compound of formula (VIII) under suitable conditions to produce a compound of formula (VII).

[chemical structure]

In another aspect, a process for preparing the GGA derivative of formula (XI) is provided. The process comprises oxidizing a compound of formula (XII) under suitable conditions to produce a compound of formula (XI).

[chemical structure]

In another aspect, a process for preparing the GGA derivative of formula (XV) is provided. The process comprises oxidizing a compound of formula (XVI) under suitable conditions to produce a compound of formula (XV).

[chemical structure]

In some embodiments, conditions suitable for oxidation of compound of formula (IV), (VIII), (XII), (XVI) include, subjecting the compound to Moffatt oxidation. As will be appreciated by one skilled in the art, Moffat oxidation is the reaction of primary and secondary alcohols by dimethyl sulfoxide (DMSO) activated with a carbodiimide, such as dicyclohexylcarbodiimide (DCC) in presence of an acid to produce an alkoxysulfonium ylide which rearranges to generate aldehydes and ketones, respectively (K. E. Pfitzner and J. G. Moffatt, J. Am. Chem. Soc., 85, 3027 (1963)). Swern Oxidation may also be used, which in some embodiments employs DMSO and oxalyl chloride, at low temperatures, as is well known to the skilled artisan.

In some embodiments, other methods suitable for oxidation of an alcohol to an aldehyde can be utilized. For example, the alcohol can be oxidized under Parikh-Doering oxidation conditions using DMSO as the oxidant, activated by the sulfur trioxide pyridine complex in the presence of alkylamine base, e.g., triethylamine. In other embodiments, the alcohol compound can be oxidized under Swern oxidation conditions using oxalyl chloride, dimethyl sulfoxide (DMSO) and an organic base, such an alkylamine base, e.g., triethylamine.

In another aspect, a process for preparing the GGA derivative of formula (IV) is provided. The process comprises reducing a compound of formula (V) under suitable conditions to produce a compound of formula (IV).

[chemical structure]

In another aspect, a process for preparing the GGA derivative of formula (VIII) is provided. The process comprises oxidizing a compound of formula (IX) under suitable conditions to produce a compound of formula (VIII).

[chemical structure]

In another aspect, a process for preparing the GGA derivative of formula (XII) is provided. The process comprises oxidizing a compound of formula (XIII) under suitable conditions to produce a compound of formula (XII).

[chemical structure]

In another aspect, a process for preparing the GGA derivative of formula (XVI) is provided. The process comprises oxidizing a compound of formula (XVII) under suitable conditions to produce a compound of formula (XVI).

[chemical structure]

As will be appreciated by one skilled in the art, suitable reducing agents for the reduction of acid of formula (V), (IX) or (XIII) include reducing hydrides, preferably aluminum hydrides or borohydrides, more preferably metal aluminum hydrides in which the metal is a group I or group II metal such as lithium, sodium, potassium, calcium, magnesium or the Particularly preferred metal aluminum hydrides include lithium aluminum hydride (LAH), sodium aluminum hydride, and mixture thereof. The reduction is typically conducted in aprotic solvents such as ethers, e.g. tetrahydrofuran or aromatic hydrocarbons e.g., benzene and toluene, at low to reflux temperature using from about 0.5 to about 3.0 moles of hydride reducing agent per mole of compound of formula (V). In one embodiment, the preferred reducing agent is lithium aluminum hydride. Suitable solvents include dioxane, toluene, diethyl ether, tetrahydrofuran (THF), dipropyl ether and the like. In one embodiment, the preferred solvent is diethyl ether or THF.

In another aspect, a process for preparing the GGA derivative of formula (V) is provided. The process comprises reacting a compound of formula (VI) under suitable conditions to produce a compound of formula (V).

[chemical structure]

In another aspect, a process for preparing the GGA derivative of formula (IX) is provided. The process comprises reacting a compound of formula (X) under suitable conditions to produce a compound of formula (IX).

[chemical structure]

In another aspect, a process for preparing the GGA derivative of formula (XIII) is provided. The process comprises reacting a compound of formula (XIV) under suitable conditions to produce a compound of formula (XIII).

[chemical structure]

In some embodiments, suitable conditions include subjecting the allylic alcohol of formula (VI), (X) or (XIV) to Johnson-Claisen rearrangement to give the γ,δ-unsaturated ester of formula (VI). In one embodiment, the compound of formula (V) is condensed with a tri-(C₁-C₆)alkyl orthoacetate and further the intermediate allyl-enol ether is rearranged, without isolation, in the presence of an acid in a reaction inert solvent. The orthoacetate utilized for the process is preferably selected from trimethyl orthoacetate and triethyl orthoacetate. In one embodiment, the orthoacetetate has the formula)CH₃—CH₂—(OR¹⁰)₃, where R¹⁰ is C₁-C₆ alkyl. In some embodiments, the acid is a weak acid, preferably a simple carboxylic acid such as a propionic acid or isobutyric acid, or an alkane or arene sulphonic acid e.g., p-toluene sulphonic acid. The process is carried out at an elevated temperature preferably the reflux temperature, under conditions where alcohol generated by the process can be removed from the reaction mixture.

In another aspect, a process for preparing the GGA derivative of formula (VI) is provided. The process comprises alkenylating a compound of formula (VII) with R³C(—)═CH₂ under suitable conditions to produce a compound of formula (VI).

[chemical structure]

In another aspect, a process for preparing the GGA derivative of formula (X) is provided. The process comprises alkenylating a compound of formula (XI) with R⁴C(—)═CH₂ under suitable conditions to produce a compound of formula (X).

[chemical structure]

In another aspect, a process for preparing the GGA derivative of formula (XIV) is provided. The process comprises alkenylating a compound of formula (XV) with R⁵C(—)═CH₂ under suitable conditions to produce a compound of formula (XIV).

[chemical structure]

R³, R⁴, and R⁵ are as defined herein above. In some embodiments, conditions suitable for the alkenylation of the aldehyde of formulas (VII), (XI), and (XV) include, contacting the compound of formula (VII) with an appropriate organometallic reagent in a reaction inert solvent. The organometallic reagent utilized for the C-alkenylation of carbonyl compounds to the allyl alcohol preferably contain magnesium halides or lithium moieties. Suitable inert solvents utilized in the process will be apparent to one skilled in the art. In one embodiment, the solvent is THF or diethyl ether.

In another aspect, a process for preparing the GGA derivative of formula (XVII) is provided. The process comprises reacting a compound of formula (XVIII) under suitable conditions to produce a compound of formula (XVII).

[chemical structure]

In some embodiments, conditions suitable for ketal, thioketal, or an oxa-thioketal (having an —O—C—S— moiety) formation include, reacting the carbonyl compound of formula (XVIII) with a suitable alcohol, alpha omega diol, a thiol, an alpha omega dithiol, or an omega hydroxy thiol solvent under acidic conditions. In one embodiment, the ester of Formula (XVIII) is reacted with a suitable alcohol such as e.g., ethylene glycol, mercaptoethanol and 1,2-dithioethanol in the presence of a suitable acid catalyst followed by azeotropic removal of water. Suitable acid catalysts include, e.g., strong mineral acids, such as sulfuric, hydrochloric, hydrofluoroboric, hydrobromic acids, p-toluenesulfonic acid, camphorsulfonic acid, methanesulfonic acid, and like. Various resins that contain protonated sulfonic acid groups are also useful as they can be easily recovered after completion of the reaction. Examples of acids also include Lewis acids. For example, boron trifluoride and various complexes of BF₃, such as e.g., BF₃ diethyl etherate. Silica, acidic-alumina, titania, zirconia, various acidic clays, and mixed aluminum or magnesium oxides can be used. Activated carbon derivatives comprising mineral acid, sulfonic acid, or Lewis acid derivatives can also be used.

In one aspect, a process for preparing the GGA derivative of formula (XX) is provided. The process comprises contacting a compound of formula (XIX) with an acid catalyst under conditions suitable for hydrolysis of compound of formula (XX) to produce a compound of formula (XIX).

[chemical structure]

In one embodiment, the conditions suitable for hydrolysis of compound of formula (II) to produce a compound of formula (I) include contacting the compound of formula (II) with an acid catalyst in a compatible solvent at a suitable temperature.

In one embodiment, the acid catalyst utilized in the process is selected from an aqueous acetic acid, formic acid, trifluoroacetic acid, sulfuric acid, hydrochloric acid, methanesulfonic acid, alkyl or aralkylsulfonic acid or a Lewis acid.

In one embodiment, the GGA prepared according to this invention is 5-trans GGA or substantially pure 5-trans GGA which is optionally free of cis GGA or is essentially free of cis GGA. In other embodiment, the GGA prepared according to this invention is 5-cis GGA or substantially pure 5-cis GGA which is optionally free of trans GGA or is essentially free of trans GGA.

The starting materials for the reactions described herein are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1 15 (John Wiley and Sons, 1991), Rodd's Chemistry of Carbon Compounds, Volumes 1 5 and Supplementals (Elsevier Science Publishers, 1989), Organic Reactions, Volumes 1 40 (John Wiley and Sons, 1991), March's Advanced Organic Chemistry, (John Wiley and Sons, 4^{th} Edition), and Larock's Comprehensive Organic Transformations (VCH Publishers Inc., 1989).

Levulinic esters, such as methyl levulinate or ethyl levulinate, can be converted to the corresponding ketal (X═Y═O), hemi-thioketal (X═O, Y═S), or dithio-ketal, (X═Y═S), by reacting with ethylene glycol, mercapto ethanol, or ethane-1,2-dithiol, under acidic conditions that facilitate removal of water. Typical acid catalysts include p-toluenesulfonic acid, or acetic acid and boron trifluoride-etherate. Solvents used for such transformations include benzene, toluene and methylene chloride. Water is removed by azeotropic distillation or by reaction with an ortho-ester such as triethyl ortho-formate or triethyl ortho-acetate. (Reaction 1)

Conversion of the ketal-ester, from Reaction 1 to the corresponding aldehyde, can be accomplished in a single step shown in Reaction 2 by reduction with a hindered active metal hydride such as di-isobutyl aluminum hydride at reduced temperatures in ether, followed by quenching with ethyl acetate to consume excess reagent. Temperatures for such reactions typically must be kept below −35° C. to minimize over-reduction to the alcohol. (Reaction 2)

Alternatively, the aldehyde, can be prepared in higher yield and greater purity in two separate steps. The first step, Reaction 3, involves complete reduction of the ester to the corresponding alcohol, with a strong reducing agent such as lithium aluminum hydride in diethyl ether or THF. This reduction is followed by oxidation of the alcohol to the aldehyde per Reaction 4, by one of the several methods listed below. (Reaction 4)

Use of chromium trioxide in pyridine for oxidation of alcohols to aldehydes is reported. Alternatively, this oxidation can be accomplished with dimethyl sulfoxide and any of a variety of dehydrating agents. Published examples include various acid chlorides, acid anhydrides, and carbodiimides.

These reactions typically require temperatures below −35° C. prevent side reactions. The method employing a sulfur trioxide-pyridine complex in the presence of triethylamine can be conducted at room temperature with minimal side reactions. (Reaction 5)

The aldehyde is reacted with 2-propenyl lithium or its Grignard equivalent, to give an allylic alcohol. This alcohol can be converted to olefinic esters in high yield with high stereoselectivity. (Reaction 6)

The product is then subjected to the transformations of Reactions 3 through 6 for two additional cycles to yield an ester. This ester is then reduced and oxidized as in Reactions 3 and 4 to give the alcohol in (Reaction 7) and the aldehyde in (Reaction 8).

The terminal olefin can be added by a Wittig reaction using propylidene triphenylphosphosphineylide, generated from the commercially available isopropyl(triphenyl)phosphonium bromide as shown in Reaction 9.

The final product is produced by hydrolysis of the ketal, hemithioketal, or the dithioketal.

From the foregoing it will be appreciated that, although specific embodiments of the invention have been described herein for purposes of illustration, various modifications may be made without deviating from the spirit and scope of the invention.

Throughout the description of this invention, reference is made to various patent applications and publications, each of which are herein incorporated by reference in their entirety.

1. A process for preparing a compound of formula (I): [chemical structure]
said process comprising:
hydrolyzing a compound of formula (II): [chemical structure]
wherein:
X and Y are each independently OR⁶, SR⁶, or X and Y together with the carbon atom they are attached to form a ring of formula: [chemical structure]
wherein each R⁶ is independently C₁-C₆ alkyl,
each X¹ and X² are independently O, or S; q is 1 or 2; each X³ is independently C₁-C₆ alkyl; t is 0, 1, 2, or 3, and
each of R¹, R², R³, R⁴, and R⁵ is independently H or C₁-C₆ alkyl or R¹ and R² together with the carbon atom they are joined to form a C₅-C₆ cycloalkyl optionally substituted with 1-3 C₁-C₆ alkyl.

2. The process of claim 1, wherein the compound of formula (II): [chemical structure]
is prepared comprising:
contacting a compound of formula (III): [chemical structure]
with a reagent of formula: [chemical structure]
wherein L is P(R^{z})₃, P(O)(R^{z})₂, SO₂R^{z}, or Si(R^{z})₃; and wherein R^{z} is a C₁-C₆ alkyl group or an aryl group;
under conditions suitable for olefination of compound of formula (III) to produce a compound of formula (II).

3. A process for preparing a compound of formula (XXI): [chemical structure]
said process comprising:
oxidizing a compound of formula (XXII): [chemical structure]
wherein:
X and Y are each independently OR⁶, SR⁶, or X and Y together with the carbon atom they are attached to form a ring of formula: [chemical structure]
wherein each R⁶ is independently C₁-C₆ alkyl,
each X¹ and X² are independently O, or S; q is 1 or 2; each X³ is independently C₁-C₆ alkyl; t is 0, 1, 2, or 3,
each of R⁷ independently is H or C₁-C₆ alkyl; and
n is 1-5,
under suitable conditions to provide a compound of formula (XXI).

4. The process of claim 3 wherein the compound of formula (XXII) [chemical structure]
is prepared comprising:
reducing a compound of formula (XXIII) [chemical structure]
under conditions suitable to provide a compound of formula (XXII).

5. The process of claim 4, wherein the compound of formula (XXIII): [chemical structure]
is prepared comprising:
contacting an orthoacetate of formula R⁷—CH₂—(OR¹⁰)₃, wherein R¹⁰ is C₁-C₆ alkyl, with a compound of formula (XXIV): [chemical structure]
wherein X, Y, and R⁷ are defined as in formula (II).

6. The process of claim 5, wherein the compound of formula (XXIV): [chemical structure]
is prepared comprising:
contacting of a compound of formula (XXV): [chemical structure]
with an anion of formula R⁷C(—)═CH₂.

7. The process of claim 1, wherein R¹-R⁵ are methyl.

8. The process of claim 3, wherein R⁷ is methyl.
